(19) United States
(12) Patent Application Publication
(10) Pub. No.: US 2018/0344796 A1
(43) Pub. Date: Dec. 6, 2018

(54) METHOD FOR TREATING FIBROMYALGIA

(71) Applicant: Margery A. Hursky, Hastings, MN (US)

(72) Inventor: Margery A. Hursky, Hastings, MN (US)

(21) Appl. No.: 15/607,751

(22) Filed: May 30, 2017

(51) Int. Cl. A61K 36/9066; A61K 36/324; A61K 36/288; A61K 36/87; A61K 31/197; A61K 31/714; A61K 36/752; A61K 9/00; A61K 9/08; A61K 9/20; A61K 36/82; A23L 2/52; A23L 33/00

(52) U.S. Cl. CPC A61K 36/9066; A23V 2002/00; A61K 36/288; A61K 36/87; A61K 31/197; A61K 31/714; A61K 36/752; A61K 9/0095; A61K 9/08; A61K 9/0019; A61K 9/20; A61K 36/82; A23L 2/52; A23L 33/40; A61K 36/324

(57) ABSTRACT

Symptoms of fibromyalgia are treated by daily administration of a regiment comprising a combination of naturally occurring ingredients selected from a group consisting of turmeric extract, boswellia extract, dandelion root, grape seed extract, green tea, a bioflavonoid complex, acetyl L-carnitine, and vitamin B12. Such ingredients are generally safe for human consumption and, when used in combination, relieve the symptoms associated with fibromyalgia.

### CROSS-REFERENCED TO RELATED APPLICATIONS

Not applicable

### STATEMENT REGARDING FEDERALLY SPONSORED RESEARCH OR DEVELOPMENT

Not applicable

### BACKGROUND OF THE INVENTION

### I. Field of the Invention

The present invention relates to the treatment of fibromyalgia. More specifically, the present invention relates to daily regiments of naturally occurring substances that may be administered to reduce or eliminate symptoms associated with fibromyalgia.

### II. Discussion of the Prior Art

Fibromyalgia affects an estimated 5.8 million Americans. Fibromyalgia is a syndrome that affects the muscles and soft tissues of the body. Symptoms include chronic, widespread musculoskeletal pain accompanied by fatigue, sleep problems, and painful tender points, sometimes referred to as trigger points. Some patients suffering from fibromyalgia also report memory problems, restless legs syndrome, bowel or bladder problems, and numbness and tingling in the extremities. Depression and anxiety are also often associated with fibromyalgia.

The cause of fibromyalgia is unknown and physicians are not in agreement regarding what should be considered the essential diagnostic criteria or even whether an objective diagnosis is possible. However, the American College of Rheumatology recommends the use of a widespread pain index and system severity scale to score symptoms that have persisted at a similar level for at least three months and that cannot be explained by another diagnosable disorder.

A variety of drugs have been used to manage the symptoms of fibromyalgia. Such drugs include pregabalin (sold under the trademark Lyrica™), duoloxetine, antidepressants, anti-seizure medications, and opioids, among others. Other non-pharmacological approaches have also been employed to treat symptoms of fibromyalgia. To date, no single intervention has been shown to be effective for all patients. Further, use of certain drugs (e.g., opioids) is discouraged due to side effects and addiction issues.

Thus, there continues to be a need for a safe and effective treatment of fibromyalgia symptoms.

### SUMMARY OF THE INVENTION

The inventor, who has long suffered from fibromyalgia, has discovered that daily administration of the following regiment of natural substances has caused her fibromyalgia symptoms to subside:

| | | |
|---|---|---|
| Turmeric extract | 1500 | mg |
| (standardized to 95% curcumin) | | |
| Boswellia extract | 500 | mg |
| Dandelion root | 1500 | mg |
| Grape seed extract | 50 | mg |
| Green tea | 50 | mg |
| Lemon bioflavonoid complex | 50 | mg |
| Acetyl L-carnitine | 500 | mg |
| Vitamin B₁₂ | 5000 | mcg |

The above listed components of the regiment may be administered together as a single dose once a day or may be administered incrementally throughout the day to better spread their beneficial effects.

This regiment works most effectively when consumed with plenty of fluids. What constitutes “plenty of fluids” will vary from person to person. Some advocate a consumption of eight 8-ounce glasses of fluid a day. Climate and weather, level of exercise, illness or other health conditions, conditions, pregnancy and breast-feeding can all contribute to the need for even greater consumption of fluids. Generally, if a person drink enough fluid so that the person rarely feels thirsty, excretes about 6⅓ cups (1.5 liters) or more of urine a day, and the urine is colorless or light yellow, fluid intake is probably adequate. A doctor, a registered dietitian, or health professional can help a person determine what constitutes “plenty of fluid” for that person.

### DESCRIPTION OF THE PREFERRED EMBODIMENT

No single drug has proven effective in the treatment of fibromyalgia. Therefore, the inventor set out to discover a regiment of natural substances that would prove to be more effective and less addictive than the drugs often used to relieve symptoma of fibromyalgia. In doing so, the inventor discovered a regiment of such natural items that has proven to reduce the inventor's own symptoms and which the inventor believes would likely relieve the symptoms of others.

The regiment discovered by the inventor includes administration of 750 mg of turmeric twice per day. Turmeric is a spice that comes from the turmeric plant that contains a yellow-colored chemical called curcumin. The turmeric used by the inventor is standardized to 95% curcuminoids. Turmeric is known to be safe since it is a spice routinely used in Asian food. It is also the main ingredient in curry. Turmeric is also an ingredient in medicines used to treat arthritis and joint pain. While others have used turmeric to treat fibromyalgia, to the inventor's knowledge turmeric has not been used in combination with the other substances discussed below which, together, comprise the inventor's regiment.

A second substance that may be included in the regiment of the present invention is boswellia extract, Boswellia is a genus of the trees in the order sapindales that produce a fragrant resin having many pharmacological uses, particularly as anti-inflammatories. 500 mg per day of boswellia extract may be included as part of the regiment of the present invention.

A third component that may be included in the regiment of the present invention is dandelion root. Like the other ingredients, dandelion is an herb. Dandelion that is been used as salad greens and in soups, wines and teas for many years. Dandelion root contains chemicals that decrease swelling and inflammation. As such, the daily regimen of the present invention may include 1500 mg of ground dandelion root.

A fourth component that may be used in the regiment of the present invention is a mixture of three ingredients, specifically equal parts of grape seed extract, green tea, and lemon bioflavonoid complex. Grape seed extract is a derivative of whole grape seed that contains organic proanthocyanidins, Vitamin E, flavonoids, and linoleic acid. Standardized extracts of grape seed have been used to treat a range of health problems related to free radical damage. Grape seed extract has also been shown to protect against bacterial infection. Flavonoids have been shown to help lower LDL (“bad”) cholesterol. Grape seed extract has also been used to reduce edema in post-surgical breast cancer patients and patients recuperating from sports injuries.

Green tea is a type of tea made from Camellia sinensis leaves that have not been allowed to wither or oxidize. Extracts of green tea have been used in traditional Chinese and Indian medicines for many years. Standardized green tea extract is 90% total polyphonols.

Lemon bioflavonoid complex comprises flavonoids which preliminary studies suggest may affect anti-inflammatory mechanisms in several ways. First, flavonoids inhibit reactive oxygen or hydrogen compounds. Second, flavonoids inhibit pro-inflammatory activity of enzymes involved in free radical production. The flavonoids work to reduce free radicals. Third, flavonoids modify intra-cellular signal pathways. Fourth procyanidins, a class of flavonoids, have been shown in preliminary research to have anti-inflammatory mechanisms. Fifth, flavonoids act as antioxidants. While the inventor has chosen to use lemon bioflavonoid complex, the reader should understand that other citrus bioflavonoid complexes may be used as a substitute for lemon bioflavonoid complex without deviating from the invention.

In one embodiment of the regiment of the present invention, 50 mg each of grape seed extract, green tea and lemon bioflavonoid complex are administered daily.

A fifth component that may be administered as part of the daily regiment of the present invention is Acetyl L-carnitine (“ALC”). ALC is naturally produced in the body. ALC is broken down in the blood into carnitine which is used by the body to transport fatty acids into the mitochondria for breakdown. Research has linked ALC with less pain and less intense pain in people with nerve problems.

Still another ingredient that may be included in the regiment of the present is Vitamin B₁₂. This vitamin plays a key role in normal functioning of the brain and nervous system. Vitamin B₁₂ deficiency is known to cause severe and irreversible damage to the brain and nervous system. Levels of Vitamin B₁₂ slightly lower than normal cause symptoms such as fatigue, lethargy, depression, poor memory, headaches, all of which are symptoms commonly associated with fibromyalgia. Various embodiments of the regiment of the present invention include daily administration of 5000 mcg of Vitamin B₁₂. Of course, similar doses of Vitamin B₁₂ can be consumed in the form of energy drinks or by eating foods known to have high concentrations of (or to be fortified with) Vitamin B₁₂. Such foods include breakfast cereals, soy products, energy bars, and meat and dairy products.

Accordingly, one embodiment of the present invention includes administration to the patient of a regiment including the following ingredients:

| | | |
|---|---|---|
| Turmeric extract | 1500 | mg |
| (standardized to 95% curcumin) | | |
| Boswellia extract | 500 | mg |
| Dandelion root | 1500 | mg |
| Grape seed extract | 50 | mg |
| Green tea | 50 | mg |
| Lemon bioflavonoid complex | 50 | mg |
| Acetyl L-carnitine | 500 | mg |
| Vitamin B₁₂ | 5000 | mcg |

Some sufferers of fibromyalgia may find sufficient relief even if the quantity of one or more of the ingredients listed above is reduce, or even if that ingredient is eliminated altogether, or if that ingredient is replaced by an equivalent ingredient. By way of example and without limitation, a citrus bioflavonoid complex other than lemon bioflavonoid complex may be employed without deviating from the invention.

The regiment described above (and variations thereof) works most effectively when consumed with plenty of fluids. What constitutes “plenty of fluids” will vary from person to person. Some advocate a consumption of eight 8-ounce glasses of fluid a day. As such, the regiment and variations thereof may also include fluid consumption of at least 60 ounces per day. Climate and weather, level of exercise and exertion, illness or other health conditions, pregnancy and breast-feeding can all contribute to the need for even greater consumption of fluids. Generally, if a person drink enough fluid so that the person rarely feels thirsty, excretes about 6⅓ cups (1.5 liters) or more of urine a day, and the urine is colorless or light yellow, fluid intake is probably adequate. A user of any variation of the regiment described above may wish to consult a doctor, a registered dietitian, or other health professional who can help a person determine what constitutes “plenty of fluid” for that person.

Further, the combination of ingredients can be administered orally in pill form (i.e., as a tablet or capsule), intravenously, or via injection. In some cases the ingredients may be administered orally in the form of a drink having a diluent in which some of the ingredients are dissolved and with which others are mixed. Various other forms of administration are available without deviating from the invention.

Likewise, if the ingredients are contained in one or more tablets and/or capsules taken orally, they may be combined so that a portion of the daily recommended dosage of each ingredient is in a single pill and multiple pills are taken throughout the day. Alternatively, the ingredients may be administered separately in differing forms.

The formulation of the regiment may also be altered to eliminated altogether (or replace with other ingredients) any of the ingredients contraindicated for a specific patient. For example, pregnant or breastfeeding women should not participate in a regiment involving ingestion of grape seed extract. Also, herbs, including those discussed above, contain components that can trigger side effects and interact with other herbs, supplements, or medications. For these reasons, the regiment (and the variations discussed herein) should be used with care, under the supervision of a health care provider qualified in the field of botanical medicine. Common side effects of herbs can include nausea, itching, dizziness, stomach upset, diarrhea, headache, sore throat, cough, and rash.

Various other modifications may be made without deviating from the invention.

What is claimed is:

1. A regiment for treating fibromyalgia comprising daily administration of at least five ingredients, each of said ingredients selected from a group consisting of:
turmeric extract, boswellia extract, dandelion root, grape seed extract, green tea, a bioflavonoid complex, acetyl L-carnitine, and vitamin B12.

2. A regiment for treating fibromyalgia comprising daily administration of turmeric extract, boswellia extract, grape seed extract, a bioflavonoid complex, acetyl L-carnitine, and vitamin B12.

3. The regiment for treating fibromyalgia of claim 2 further comprising daily administration of dandelion root and green tea.

4. The regiment for treating fibromyalgia of claim 2 wherein the quantities of turmeric extract, boswellia and acetyl L-carnitine administered daily are each at least 500 mg.

5. The regiment for treating fibromyalgia of claim 3 wherein the quantities of turmeric extract and dandelion root administered daily is each as at least 1500 mg.

6. The regiment for treating fibromyalgia of claim 2 wherein the turmeric extract is standardized to 97% curcuminoids.

7. The regiment for treating fibromyalgia of claim 2 wherein the bioflavonoid complex is a citrus bioflavonoid complex.

8. The regiment for treating fibromyalgia of claim 2 wherein the citrus bioflavonoid complex is a lemon bioflavonoid complex.

9. The regiment for treating fibromyalgia of claim 2 wherein the daily administration of said turmeric extract, boswellia extract, grape seed extract, a bioflavonoid complex, acetyl L-carnitine, and vitamin B12 is in the form of a drink.

10. The regiment for treating fibromyalgia of claim 2 wherein the daily administration of said turmeric extract, boswellia extract, grape seed extract, a bioflavonoid complex, acetyl L-carnitine, and vitamin B12 is in the form of a solution adapted to by administered either intravenously or by injection.

11. The regiment for treating fibromyalgia of claim 2 wherein the daily administration of said turmeric extract, boswellia extract, grape seed extract, a bioflavonoid complex, acetyl L-carnitine, and vitamin B12 is in the form of a mixture adapted to be administered either in pill form.

12. The regiment for treating fibromyalgia of claim 2 further including consumption of at least 60 ounces of fluid each day.
